(19) United States
(12) Patent Application Publication
(10) Pub. No.: US 2020/0315829 A1
(43) Pub. Date: Oct. 8, 2020

(54) INDUCING WEIGHT LOSS BY APPLICATION OF ENERGY

(71) Applicant: RAMBAM MED-TECH LTD., Haifa (IL)

(72) Inventors: Ilana Zehavit SCHLESINGER, Netanya (IL); Alon SINAI, Koranit (IL)

(21) Appl. No.: 16/835,608

(22) Filed: Mar. 31, 2020

Related U.S. Application Data
(60) Provisional application No. 62/829,114, filed on Apr. 4, 2019.

(51) Int. Cl. A61F 5/00; A61N 7/02; A61B 5/00; A61B 5/055; A61B 17/32

(52) U.S. Cl. CPC A61F 5/0003; A61N 7/02; A61B 5/0036; A61N 2007/0026; A61B 17/320068; A61B 2017/320069; A61B 5/055

(57) ABSTRACT

A method is provided for inducing weight loss in a subject. The method includes using an energy source, applying energy to thalamic nuclei of a subject, modifying at least a portion of the thalamic nuclei by applying the energy, and inducing weight loss in the subject.

### CROSS REFERENCE TO RELATED APPLICATIONS

This application claims the benefit of priority of U.S. Provisional Patent Application No. 62/829,114, filed Apr. 4, 2019 titled: “INDUCING WEIGHT LOSS BY APPLICATION OF ENERGY”, the contents of which are all incorporated herein by reference in their entirety.

### FIELD OF THE INVENTION

Applications of the present invention relate generally to methods for reducing weight. Some applications of the present invention relate more specifically to modification or stimulation of brain tissue for reducing weight.

### BACKGROUND OF THE INVENTION

Focused ultrasound (FU) and High Intensity Focused Ultrasound (HIFU) are non-invasive, image-guided surgical technologies that use ultrasound energy to target specific areas of the brain and body in the treatment, or investigation, of safety and efficacy in various diseases including; essential tremor and Parkinson's disease. Ultrasound waves pass through tissue and bone and converge in the tissue to heat the tissue to temperatures sufficient to cause thermal ablation, destroying the target cells. At lower intensities, the same technology can be used to stimulate the tissue.

Magnetic Resonance Imaging (MM) uses magnetic fields and radio waves to form detailed images of the anatomy as well as thermometry. The technique is widely used in hospitals for medical diagnosis, staging of disease and follow-up.

Mill guided focused ultrasound (MRgFUS) combines these two technologies of High intensity focused ultrasound and Magnetic Resonance Imaging (MRI).

### SUMMARY OF THE INVENTION

Some applications of the present invention provide methods for thermal ablation or stimulation of targeted structures in the brain in a controlled manner to induce weight loss in a subject. In particular, some applications of the present invention provide controlled application of thermal energy to the thalamic nuclei to induce weight loss in the subject. For example, High Intensity Focused Ultrasound (typically MRI guided focused ultrasound (MRgFUS)), is applied through an intact skull of the subject, such that the ultrasound rays are concentrated on the thalamic nuclei causing an ablative focal lesion in the thalamus, and weight loss is induced in the subject. More specifically, some applications of the present invention provide a method for suppressing a subject's appetite for carbohydrates (e.g., sweets) by causing an ablative lesion in thalamus, typically by applying MRI guided focused ultrasound (MRgFUS) to the thalamic nuclei.

Some applications of the present invention provide methods for brain tissue modification, modification of brain tissue activity and brain tissue stimulation. For some applications, modification comprises thermal ablation or stimulation of targeted structures in the brain in a controlled manner to induce weight loss in a subject. For some applications modification of tissue comprises modification of secretion of neurotransmitters or hormones, tissue affinity and transmission in neuronal tracts. In particular, some applications of the present invention provide a method including applying energy to thalamic nuclei of a subject, creating an ablative lesion or stimulating in at least a portion of the thalamic nuclei, and inducing weight loss in the subject.

In some applications, the method includes using an energy source and generating the energy. In some applications, the energy source includes a high intensity focused ultrasound (HIFU) source, and wherein applying the energy includes applying the energy as high intensity focused ultrasound (HIFU) energy. In some applications, applying the energy further includes applying the energy under guidance of Magnetic Resonance Imaging (MRI).

In some applications, inducing weight loss includes inducing weight loss by suppressing the subject's appetite and food craving and food seeking behavior for food and sweet carbohydrates. Suppressing the subject's appetite for inducing weight loss in some subjects includes suppressing the appetite of the subject for eating between meals, food seeking behavior, emotional eating or craving sweet carbohydrates, while generally not suppressing the appetite for sustenance meals.

In some applications, inducing the weight loss includes reducing at least 3% of a weight of the subject. Inducing the weight loss includes inducing the weight loss over a period from the application of the energy at the end of which a weight loss of at least 3% of a weight of the subject is achieved.

Some applications of the present invention provide methods of applying energy to tissue of at least a portion of one or more thalamic nuclei and/or neuronal tracts in propinquity to the thalamic nucleus of a subject, modifying the tissue and inducing weight loss in the subject. For some applications, modifying the tissue includes creating an ablative lesion and/or stimulating at least a portion of the thalamic nucleus and/or neuronal tracts in propinquity to the thalamic nucleus. For some applications, the portion of the thalamic nucleus includes a Weight Reduction Inducing Region (WRIR) which is in propinquity to the thalamic VIM.

For some applications, the method includes applying the energy to the tissue of the thalamic nucleus and/or neuronal tracts in propinquity to the thalamic nucleus within a radius of less than 10 mm of the thalamic VIM. For some applications, the energy source includes a high intensity focused ultrasound (HIFU) source, and wherein applying the energy includes applying the energy as high intensity focused ultrasound (HIFU) energy. For some applications, applying high intensity focused ultrasound (HIFU) energy includes applying energy of between 100 joules up to 35,000 Joules.

For some applications, the method includes applying the energy for a period of time between 5 and 40 seconds. For some applications, the method includes applying the energy for a period of time between 0.5 seconds and 60 minutes. For some applications, the method includes applying the energy to achieve a temperature at the target tissue of between 50-60° C. For some applications, the method includes applying the energy to achieve a temperature at the target tissue of between 40-50° C.

For some applications, the method includes applying the energy in a form of sonications. For some applications, the method includes generating between 1 and 28 sonications per treatment. For some applications, the method includes generating between 1 and 3000 low energy sonications per treatment. For some applications, the method includes applying the energy under guidance of Magnetic Resonance Imaging (MM).

For some applications, inducing weight loss includes selectively suppressing the subject's appetite. For some applications, selectively suppressing the subject's appetite includes suppression of food craving and food seeking behavior for carbohydrates and sweets while maintaining appetite for sustenance. For some applications, inducing the weight loss includes reducing at least 3% of a weight of the subject. For some applications, inducing the weight loss includes inducing the weight loss over a period of at least three months from the application of the treatment. For some applications, the method includes reducing required insulin dose as well as other medications to treat diabetics, hypertension, and hyper lipidemia.

### DETAILED DESCRIPTION OF THE INVENTION

In accordance with some aspects of the present invention, a method is provided for applying energy to the thalamic nuclei of a subject, creating either an ablative lesion or stimulation in at least a portion of the thalamic nuclei by applying the energy, and inducing weight loss in the subject, by creating the ablative lesion or stimulation of the tissue.

In accordance with some aspects of the present invention, the energy is applied as high intensity focused ultrasound (HIFU) energy.

In accordance with some aspects of the present invention, the high intensity focused ultrasound energy is applied under guidance of MM through an intact skull of the subject.

In accordance with some aspects of the present invention, a method is provided for inducing weight loss in a subject by inducing a change in eating habits of the subject.

In accordance with some aspects of the present invention, a method is provided for inducing weight loss in a subject by selectively suppressing the subject's appetite includes suppression of food craving and food seeking behavior for carbohydrates and sweets while maintaining appetite for sustenance.

### Methods Used in Some Applications of the Present Invention

A series of protocols are described hereinbelow which may be used separately or in combination, as appropriate, in accordance with applications of the present invention. It is to be appreciated that numerical values are provided by way of illustration and not limitation. Typically, but not necessarily, each value shown is an example selected from a range of values that is within 10% of the value shown. Similarly, although certain steps are described with a high level of specificity, a person of ordinary skill in the art will appreciate that other steps may be performed, mutatis mutandis.

While treating patients suffering from essential tremor (ET), and more specifically ET patients who underwent MRgFUS VIM thalamotomy, authors of this disclosure were surprised to discover that at least a portion of patients suffering from ET and treated by application of ablative focused ultrasound to the thalamic nuclei (versus the MRgFUS VIM thalamotomy) experienced at least partial reduction in appetite and more specifically a reduction in affinity to carbohydrates e.g., sweets.

Although the experimental data described herein is shown for subjects diagnosed with ET (essential tremor), it is to be understood that the scope of the present invention includes treating healthy subjects or subjects suffering from conditions other than ET in accordance with techniques described herein.

In accordance with some applications of the present invention, the following methods were applied:

### Obtaining Subject and Control Populations

Data was collected from ET (essential tremor) subjects who underwent MRgFUS VIM thalamotomy at the Rambam Health Care Campus, Haifa, Israel. The information regarding variation in body weight was collected from the outpatient charts as well as from interviewed ET patients who underwent MRgFUS VIM thalamotomy at the Rambam Health Care Campus, Haifa, Israel to evaluate changes from baseline in bodyweight throughout the follow-up. The patients were divided into two groups: those who lost more than 3% body weight versus those who did not. Among others, the collected data comprised of weight, height, self-reported food seeking behavior and hypogeusia.

The control population comprised ET subjects that did not experience weight loss. This retrospective study was approved by the institutional review board of Rambam Health Care Campus (RMB 0404-17).

The diagnosis of ET was confirmed by a neurologist specializing in movement disorders (I.S.) according to accepted criteria. Refractory tremor was considered as a disabling tremor after ample treatment trials. All subjects had no contraindications for the procedure including but not limited to significant cognitive decline, current anticoagulant or anti-aggregant therapy, brain tumors, vascular malformations, significant unstable medical conditions and contraindications for MR, including claustrophobia.

### Assessment of Tremor in Subject Population

Tremor was assessed using the Clinical Rating Scale for Tremor (CRST) score (ranging from 0-160 points, higher scores indicating greater disability) (10). A clinically significant tremor was defined as a score of 2 or more on the postural or action item of the CRST (ranging from 0 to 4), as well as substantial disability in the performance of at least two daily activities from the disability subsection of the scale. We also calculated CRST scores in the treated side as well (maximum overall score, 32).

Quality of life in ET subjects was measured by the Quality of Life in Essential Tremor Questionnaire (QUEST) (ranging from 0-120, higher scores indicate lower quality of life). Adverse events were rated according to the Clavien-Dindo criteria (range 1-5, higher score representing a more severe event).

The subject population included fifty-one patients who underwent MRgFUS for ET. From this patient cohort, eight patients were lost to follow-up, three patients were followed for less than three months, weight-associated reports from three patients were inconclusive and one patient lost weight due to metastatic cancer. The remaining thirty six patients were followed for 3-60 months (median 36 months, average 31.4±20.1 months) from the procedure (Table A). Eleven patients reported significant weight loss (31%) after undergoing MRgFUS (Table B). Patients lost between 5-15% of their weight (average 9.4±3.7), 4-11 kg (average 7.5±3.3 kg). The weight loss persisted during follow-up of 3-60 months (mean 31.4±21.1) (Table B) In the group of patients that lost weight after MRgFUS, the weight loss was significant (82.8±12.9 kg before the procedure, compared with 75.7±12.8 kg after the procedure; p<0.0001). Similarly, in this group of patients, the BMI after the procedure was significantly lower than before the procedure (28.5±2.7 kg/m² before the procedure, compared to 26.1±2.2 kg/m² after the procedure; p<0.0001). No patient became underweight due to weight loss.

Patients reported a temporal correlation between the time of the MRgFUS treatment and weight loss, though none of the patients linked the weight loss to the MRgFUS on their own. All eleven patients reported that they did not make any attempts to lose weight and did not intentionally modify their diet. Nine of the patients reported reduced food seeking behavior. The patients reported that after a meal they felt satiety and had stopped eating between meals.

TABLE A

| | | | | | | | | | Reduced | | |
|---|---|---|---|---|---|---|---|---|---|---|---|
| | | Follow- | | Weight | Weight | Weight | BMI | BMI | food | | Adverse |
| | | up | Height | Before | loss | loss | Before | after | seeking | | events of |
| Age | Sex | (Months) | (cm) | (Kg) | (Kg) | % | (Kg/m²) | (Kg/m²) | behavior | Hypogeusia | MRgFUS |
| 70 | male | 60 | 175 | 93 | 5 | 5 | 30.4 | 28.7 | + | − | |
| 77 | male | 55 | 171 | 73 | 4 | 5 | 25.0 | 23.6 | UK | | |
| 77 | female | 52 | 152 | 68 | 9 | 13 | 29.4 | 25.5 | UK | + | |
| 74 | male | 48 | 162 | 65 | 5 | 8 | 24.8 | 23.2 | + | + | Ataxia- |
| | | | | | | | | | | | transient |
| 78 | female | 37 | 162 | 80 | 10 | 13 | 30.5 | 26.7 | UK | | |
| 66 | male | 36 | 176 | 97 | 14 | 14 | 31.3 | 26.8 | + | − | |
| 68 | male | 24 | 170 | 90 | 10 | 11 | 31.1 | 27.7 | + | − | Right |
| | | | | | | | | | | | lower lip |
| | | | | | | | | | | | numbness- |
| | | | | | | | | | | | persistent |
| 76 | male | 18 | 178 | 100 | 7 | 7 | 31.6 | 29.3 | + | − | Ataxia- |
| | | | | | | | | | | | transient |
| 69 | male | 6 | 171 | 73 | 4 | 6 | 25.0 | 23.6 | + | + | Right |
| | | | | | | | | | | | lower lip |
| | | | | | | | | | | | numbness- |
| | | | | | | | | | | | transient |
| 62 | male | 6 | 160 | 76 | 11 | 15 | 29.7 | 25.4 | − | − | Ataxia- |
| | | | | | | | | | | | transient |
| 79 | male | 3 | 170 | 70 | 4 | 6 | 24.2 | 22.8 | + | + | |

TABLE B

| | | Patients without | Patients with |
|---|---|---|---|
| | | significant | significant |
| | All Patients | weight loss | weight loss |
| | 36 | 25 | 11 |
| Age | 72.7 ± 5.9 | 72.9 ± 6.0 | 72.2 ± 5.3 |
| Mean ± SD (range) | (62-84) | (62-84) | (62-79) |
| Gender | 22 | 13 | 9 |
| Male (%) | (61) | (52) | (82) |
| Height (cm) | 165.9 ± 8.4 | 165 ± 8.6 | 167.7 ± 7.9 |
| Mean ± SD (range) | (150-188) | (150-188) | (152-178) |
| Weight (kg) | 73.4 ± 13.0 | 70.2 ± 12.3 | 81.5 ± 11.9* |
| Mean ± SD (range) | (50-110) | (50-110) | (65-100) |
| BMI (kg/m²) | 26.6 ± 4.2 | 25.8 ± 4.4 | 28.9 ± 2.7 |
| Mean ± SD (range) | (19.9-43.0) | (19.9-43.0) | (24.2-31.6)** |

### Imaging and Treatment

All subjects underwent MRgFUS thalamotomy using a focused ultrasound system (650 kHz system, ExAblate®, Insightec, Haifa, Israel) for VIM ablation.

Initial target coordinates of the ventral intermediate nucleus (VIM) were at 25% of the AC-PC distance anterior to the PC and 14 mm lateral to the AC-PC line. VIM thalamotomy was performed contralateral to the more disabling side and according to the preference of the subject.

On the day of the procedure, the subject's head was shaven completely. A stereotactic frame (CRW Integra Plainsboro, N.J.) was fixed to the skull and an elastic silicone diaphragm affixed to the head. The subject's head was attached to the focused ultrasound 1024 US transducers helmet. A T2 weighted MRI scan was performed for planning reassessment.

MRgFUS sonications were performed using a 3-Tesla MRI (GE) and a focused ultrasound system (ExAblate Neuro, Insightec).

The method of treatment included applying high intensity focused ultrasound (HIFU) energy from a high intensity focused ultrasound (HIFU) source to tissue of a thalamic nucleus and/or neuronal tracts in propinquity to the thalamic nucleus within a radius of less than 10 mm of the thalamic VIM defined as a Weight Reduction Inducing Region (WRIR).

As explained in greater detail elsewhere herein, the method included applying energy to tissue of at least a portion of a thalamic nucleus and/or neuronal tracts in propinquity to the thalamic nucleus of a subject, modifying the tissue and inducing weight loss in the subject. For some applications, modifying the tissue includes creating an ablative lesion and/or stimulating at least a portion of the thalamic nucleus and/or neuronal tracts in propinquity to the thalamic nucleus.

Treatment included a gradual increase in total energy either by an increased intensity or by longer sonication durations. Sonications were stopped when adequate control of tremor was achieved, with the temperature reaching no more than 59° C.

The intensity was increased from 900 joules up to 35,000 Joules (average of 16500±9250 J). The level of applied energy was determined by the temperature measured at the target. The goal was to achieve a temperature of 55-60° C. (average of 56.5±2.4° C.) with a minimal duration of 9 seconds and maximal duration of 39 seconds (average of 18.7±7.8). The average number of sonications was 20.1±6.8.

All subjects underwent pre-procedural MM and CT. All MRI studies were performed using a 3 Tesla system (MR750, GE Milwaukee, USA). CT images were used to assess ratio between the bone and the bone marrow of the skull, skull density ratio (SDR) using data from the Exablate® console. An SDR of 0.3 and above was considered suitable for treatment. Information regarding skull volume available for sonication, available skull surface and number of elements used during treatment were gathered from the Exablate® console as well.

All subjects underwent post-procedural MM. All MM studies were performed using a 3 Tesla system (MR750, GE Milwaukee, USA). The average lateral-medial diameter of the lesions was 8.5±1.4 mm in patient that lost weight, compared with 6.3±1.3 mm in those who did not lose weight (P<0.0002) and the average anterior-posterior diameter of the lesion was 6.8±1.2 mm in patients that lost weight, compared with 5.4±1.1 mm in the control group (P<0.02). Lesion volume was measured from T2 SPGR image using CarestreamClinetpacs software (Carestream Health Inc., 2014). MRI images of subjects that reported weight loss and other food-related behavior changes were compared to age and sex matched subjects, from the cohort of subjects that underwent MRgFUS, that reported no such change.

### Experimental Data

The experiments described hereinbelow were performed by the inventors in accordance with applications of the present invention and using the techniques described hereinabove.

Procedure

The procedure was performed in three stages:

(i) The first stage, included sonication at very low intensity, to align the precise localization in the sagittal, axial and coronal planes. When needed the sonication focus was adjusted for precision of less than 0.2 mm. Typically the temperature at this stage reached 41-46° C.

(ii) The second stage, involved sonication at increasing intensity, in order to verify temporary effect on tremor and absence of side effects. If side effects occurred the sonication focus was moved in 0.1 mm steps until resolution of the side effects was achieved. Typically, temperature at this stage reached 46-50° C.

(iii) The third stage, the treatment stage, included gradual increase in total energy (a combination of intensity and duration of sonication). Sonication was stopped when adequate control of tremor achieved, when temperature reached 55-60° C. or when side effects were unbearable. After each sonication at the temperatures exceeding 48° C. the patient was examined and evaluated for treatment efficacy and side effects.

Thirty-six subjects underwent MRgFUS VIM thalamotomy by application of ablative energy to the VIM thalamus, in accordance with the techniques described with reference to “Imaging and Treatment”. Eleven of the thirty-six treated patients experienced weight loss (nine male patients and 2 female patients). Weight loss parameters for the eleven treated subjects was reported as set forth in Table A.

As shown, weight loss was reported in a period of time ranging from 3-60 months following application of the energy to the VIM thalamus, in accordance with some applications of the present invention. In patient that reported weight loss the weight loss typically comprised a reduction of between 5-15% of the subject weight, over the follow-up period.

Therefore, applications of the present invention comprise using an energy source, applying energy to thalamic nuclei of a subject to create a lesion in at least a portion of the thalamic nuclei by applying the energy. Additionally, applications of the present invention provide inducing weight loss in the subject by applying the energy with or without creating a lesion in the thalamus.

For some applications, applying the energy as disclosed elsewhere herein comprises applying MRgFUS sonications to the brain through an intact skull. In some cases, application of the energy involves multiple sonications, starting with low-energy sonications raising the temperature gradually. Multiple such treatments will eventually sculpt the final lesion in the brain. Since the thalamic Vim nucleus boundaries are not visible on MRI, this procedure is dependent on meticulous tenths of a millimeter movements of the focal point from one sonication to another. For subjects suffering from ET, application of positive feedback from the subject (until a therapeutic response is observed without undesired side effects for treatment of the ET) indicates thalamic nuclei ablation sufficient for inducing weight loss. For some applications, ablation or stimulation in and around portion of the thalamus will eventually induce weight loss.

For some applications the method comprises brain tissue modification, modification of brain tissue activity and brain tissue stimulation manner to induce weight loss in a subject. For some applications, modification comprises modification of secretion of neurotransmitters or hormones, tissue affinity and transmission in neuronal tracts.

For some applications, inducing weight loss in the subject comprises inducing a change in eating habits of the subject. For example, inducing weight loss in some subjects comprises selectively suppressing the appetite of the subject for eating between meals, food seeking behavior, emotional eating or craving sweet carbohydrates, while generally not suppressing the appetite of the subject for meals.

In some applications, reduced sugar intake (e.g., reduced consumption of sweets) coincides with reduced insulin dose as well as other medications to treat diabetics, hypertension, and hyperlipidemia.

As seen in table A. Hence, in some applications application of the present invention comprising using an energy source, applying energy to thalamic nuclei of a subject to create a lesion in at least a portion of the thalamic nuclei by applying the energy may reduce insulin dose requirement in diabetics.

It is noted that in accordance with some applications of the present invention, energy may be applied to the thalamus by an energy source other than ultrasound (e.g., RF, laser, cryo and/or electromagnetic energy such as ultraviolet). It is noted that in accordance with some applications of the present invention, energy may be applied to the thalamus by ultrasound without creating a lesion (e.g., by stimulation).

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

What is claimed is:

1. A method comprising:
applying energy to tissue of at least a portion of at least one thalamic nucleus of a subject;
modifying the tissue or the tissue activity; and
inducing weight loss in the subject.

2. The method according to claim 1, wherein modifying the tissue comprises creating an ablative lesion and/or stimulating at least a portion of a thalamic nucleus.

3. The method according to claim 1, wherein modifying the tissue comprises creating an ablative lesion and/or stimulating at least a portion of neuronal tracts in propinquity to the thalamic nuclei.

4. The method according to claim 1, wherein modifying the tissue activity comprises one or more of modification of secretion of neurotransmitters or hormones, tissue affinity and transmission in neuronal tracts.

5. The method according to claim 1, wherein the portion of the thalamic nucleus comprises a Weight Reduction Inducing Region (WRIR).

6. The method according to claim 1, wherein the Weight Reduction Inducing Region (WRIR) is in propinquity to the thalamic VIM.

7. The method according to claim 1, wherein applying the energy to the tissue of the thalamic nucleus within a radius of less than 10 mm of the thalamic VIM.

8. The method according to claim 1, wherein said energy source comprises a high intensity focused ultrasound (HIFU) source, and wherein applying the energy comprises applying the energy as high intensity focused ultrasound (HIFU) energy.

9. The method according to claim 8, wherein applying high intensity focused ultrasound (HIFU) energy comprises applying energy of between 100 joules up to 35,000 Joules.

10. The method according to claim 8, wherein applying the energy for a period of time between 5 and 40 seconds.

11. The method according to claim 8, wherein applying the energy for a period of time between 0.5 second and 60 minutes.

12. The method according to claim 8, wherein applying the energy to achieve a temperature at the target tissue of between 55-60° C.

13. The method according to claim 8, wherein applying the energy to achieve a temperature at the target tissue of between 40-50° C.

14. The method according to claim 8, wherein applying the energy in a form of sonications.

15. The method according to claim 7, wherein applying the energy in a form of sonications and generating between 1 and 28 sonications per treatment.

16. The method according to claim 8, wherein applying the energy in a form of sonications and generating between 1 and 3000 low energy sonications per treatment.

17. The method according to claim 1, wherein applying the energy under guidance of Magnetic Resonance Imaging (MM).

18. The method according to claim 1, wherein inducing weight loss comprises selectively suppressing the subject's appetite.

19. The method according to claim 18, wherein selectively suppressing the subject's appetite comprises suppression of food craving and food seeking behavior for carbohydrates and sweets while maintaining appetite for sustenance.

20. The method according to claim 1, wherein inducing the weight loss comprises reducing at least 3% of a weight of the subject.

21. The method according to claim 15, wherein inducing the weight loss comprises inducing the weight loss over a period of at least three months from the application of the treatment.
